(19) United States
(12) Patent Application Publication
(10) Pub. No.: US 2012/0128685 A1
(43) Pub. Date: May 24, 2012

(54) USE OF HIGH-DOSE OXAZAPHOSPHORINE DRUGS IN COMBINATION WITH MONOCLONAL ANTIBODIES FOR TREATING IMMUNE DISORDERS

(75) Inventors: Robert A. Brodsky, Brooklandville, MD (US); Richard J. Jones, Baltimore, MD (US); Francis E. O'Donnell, JR., Longboat Key, FL (US); Susan Bonitz, Flemington, NJ (US); Carlos Santos, Tampa, FL (US); Adam Kaplin, Baltimore, MD (US)

(73) Assignees: Accentia Biopharmaceuticals, inc., Tampa, FL (US); The Johns Hopkins University, Baltimore, MD (US)

(21) Appl. No.: 13/240,465

(22) Filed: Sep. 22, 2011

Related U.S. Application Data
(63) Continuation of application No. 13/071,188, filed on Mar. 24, 2011, now abandoned.
Application No. 13/071,188 is a continuation of application No. 12/851,110, filed on Aug. 5, 2010, now abandoned.
Application No. 12/851,110 is a continuation of application No. 12/637,177, filed on Dec. 14, 2009, now abandoned.
Application No. 12/637,177 is a continuation of application No. 12/434,125, filed on May 1, 2009, now abandoned.
Application No. 12/434,125 is a continuation of application No. PCT/US07/81614, filed on Oct. 17, 2007.
(60) Provisional application No. 60/856,698, filed on Nov. 3, 2006.

(51) Int. Cl. A61K 39/395; A61P 37/00

(52) U.S. Cl. 424/153.1

(57) ABSTRACT

The present invention relates to methods of treating an autoimmune disorder, using a lymphocytotoxic but hematopoeitic cell sparing high-dose pulsed amount of an oxazaphosphorine drug in combination with immune therapeutics such as, for example, monoclonal antibodies that selectively bind B-cell specific antigens.

### BACKGROUND

Autoimmune diseases afflict more than 8 million people in the U.S. alone. Autoimmunity usually occurs when the lymphocytes, which are designed to defend the body against infections and foreign agents, start attacking one or more of the body's tissues or organs. Examples of autoimmune diseases include, but are not limited to, systemic lupus erythematosus, rheumatoid arthritis, severe aplastic anemia, multiple sclerosis, autoimmune hemolytic anemia, autoimmune neurologic diseases, type I diabetes, Grave's disease, Crohn's disease, myasthenia gravis, myositis, Raynaud's phenomenon, autoimmune thrombocytopenia, chronic hepatitis and antiphospholipid syndrome.

The conventional treatment for many autoimmune diseases includes the systemic use of anti-inflammatory drugs and potent immunomodulatory agents, such as, for example, steroids, inhibitors of inflammatory cytokines, or glatiramer acetate (COPAXONE). However, despite their profound effect on immune responses, these therapies are often unable to induce clinically significant remissions in many patients.

In more recent years, researchers have contemplated the use of stem cells for the treatment of autoimmune diseases, in particular, hematopoietic stem cell transplant therapy (HCST). The rationale is to destroy the mature, long-lived and auto-reactive immune cells and to transplant a new properly functioning immune system into the patient with the hope of eliciting a remission of the autoimmune disease. However, by its very nature, HSCT is a very risky procedure and for the duration of the recovery phase, until the immune system is reconstituted, transplant recipients undergo a period of dramatically increased susceptibility to bacterial, fungal and viral infections, making this a high-risk therapy. Further, these patients often require extended or life-long immunosuppressive therapy because of re-establishment of the disease caused by the cells that are transplanted and in some instances, onset of graft versus host disease.

High-dose cyclophosphamide (for example, 50 mg/kg/day×4 days) has also been used for the treatment of certain autoimmune diseases such as, for example, severe aplastic anemia. Additionally, while low to intermediate doses of cyclophosphamide have been used in an attempt to treat other autoimmune diseases, its use is limited due to the various undesirable side effects. For example, administration of oral daily cyclophosphamide is currently one of the most effective, if not the most, immunosuppressive therapy for pemphigus vulgaris. However, the toxicity of cyclophosphamide, especially when used in low to intermediate doses over an extended period of time, has limited its use for patients with severe disease who are not responsive to or unable to tolerate nonalkylating agents.

B-lymphocyte depletion therapy has also been explored in a wide range of autoimmune diseases. See, e.g., Edwards et al., Bioche, Soc. Trans. 30:824-828 (2002). For example, immune therapeutics resulting in depletion of B-cells such as, for example, an anti-CD-20 monoclonal antibody, rituximab, have raised the hope of new therapeutics for autoimmune diseases. These therapeutics are generally less toxic and well tolerated by most patients. However, immune tolerance presents a major barrier to the use of immune therapeutics such as, for example, rituximab.

Therefore, there is a need to identify agents/therapies which can be used for breaking immune tolerance in patients, thereby to facilitate the use of less toxic therapies for the treatment of immune disorders such as, for example, B-cell depleting immune therapeutics.

### SUMMARY

This disclosure is based, at least in part, on the discovery that a lymphocytotoxic but hematopoeitic cell-sparing high-dose pulsed amount of an oxazaphosphorine drug such as, for example, cyclophosphamide, can be used for breaking immune tolerance in a patient, thereby to facilitate the use of immune therapeutics such as, for example, antibodies that selectively bind a B-cell specific antigen, in the treatment of immune disorders including for example, various autoimmune disorders.

In one aspect of the present invention, a method for eliminating or substantially reducing an autoimmune disorder in a subject is provided. The method includes administering to the subject in need thereof, a lymphocytotoxic but hematopoeitic cell sparing high-dose pulsed amount of an oxazaphosphorine drug, such that the subject's immune system reconstitutes without stem cell transplantation, and administering an effective amount of a monoclonal antibody that selectively binds a B-cell specific antigen, thereby to eliminate or substantially reduce the autoimmune disorder in the subject.

An antibody that may be used in the method of the invention is understood to be any monoclonal antibody which selectively binds to an antigen expressed on a B-cell. Exemplary antigens include but are not limited to CD3d, CD5, CD6, CD9, CD19, CD20, CD21, CD22, CD23, CD24, CD27, CD28, CD37, CD38, CD40, CD45, CD46, CD48, CD53, CD69, CD70, CD72, CD73, CD79a, CD79b, CD80, CD81, CD83, CD85a, CD85d, CD85e, CD85h, CD85i, CD85j, CD85k, CD86, CD96, CD98, CD100, CD121b, CD124, CD127, CD132, CD150, CD152, CD154, CD157, CD166, CD169, CD179a, CD179b, CD180, CD185, CD196, CD197, CD205, CDw210a, CD213a1, CD257, CD267, CD268, CD269, CD274, CD275, CD276, CD278, CD279, CD300a, CD300c, CD307, CD314, CD316, CD317, CD319, CD320, CDw327, and CD331.

In some embodiments, a method of eliminating or substantially reducing an autoimmune disorder in a subject includes administering a lymphocytotoxic but hematopoeitic cell-sparing high-dose pulsed amount of an oxazaphosphorine drug to the subject, such that the subject's immune system reconstitutes without stem cell transplantation, and administering an effective amount of a monoclonal antibody which selectively binds CD-20 to the subject, thereby to eliminate or substantially reduce the autoimmune disorder.

In some embodiments, a method of eliminating or substantially reducing an autoimmune disorder in a subject includes administering a lymphocytotoxic but hematopoeitic cell-sparing high-dose pulsed amount of an oxazaphosphorine drug to the subject, such that the subject's immune system reconstitutes without stem cell transplantation, and administering an effective amount of a monoclonal antibody which selectively binds CD-22 to the subject, thereby to eliminate or substantially reduce the autoimmune disorder.

In some embodiments of the methods according to the invention, a subject is administered a lymphocytotoxic but hematopoetic cell-sparing high-dose pulsed amount of an oxazaphosphorine drug, an effective amount of a monoclonal antibody that selectively binds CD-20 and an effective amount of a monoclonal antibody that selectively binds CD-22.

In some embodiments, a method of treating an immune disorder includes the step of administering an effective amount of granulocyte colony stimulating factor to the subject. In certain embodiments, a method of treating an autoimmune disease additionally includes the step of administering an effective amount of at least one antimicrobial agent to the subject. In certain embodiments, a method of treating an autoimmune disease additionally includes the step of administering an effective dose of platelets to the subject. In one embodiment, an effective amount of platelets is an amount which results in a platelet count of at least 10,000 platelets/mm³. In certain embodiments, a method of treating an autoimmune disease additionally includes the step of administering an effective dose of red blood cells to the subject. A method of treating an autoimmune disease, as described herein, may include any one, two, three, or all four of these additional steps.

Exemplary autoimmune diseases which may be treated using the methods of the invention include, but are not limited to, AIDS-associated myopathy, AIDS-associated neuropathy, Acute disseminated encephalomyelitis, Addison's Disease, Alopecia Areata, Anaphylaxis Reactions, Ankylosing Spondylitis, Antibody-related Neuropathies, Antiphospholipid Syndrome, Autism, Autoimmune Atherosclerosis, Autoimmune Diabetes Insipidus, Autoimmune Endometriosis, Autoimmune Eye Diseases, Autoimmune Gastritis, Autoimmune Hemolytic Anemia, Autoimmune Hemophilia, Autoimmune Hepatitis, Autoimmune Interstitial Cystitis, Autoimmune Lymphoproliferative Syndrome, Autoimmune Myelopathy, Autoimmune Myocarditis, Autoimmune Neuropathies, Autoimmune Oophoritis, Autoimmune Orchitis, Autoimmune Thrombocytopenia, Autoimmune Thyroid Diseases, Autoimmune Urticaria, Autoimmune Uveitis, Autoimmune Vasculitis, Behcet's Disease, Bell's Palsy, Bullous Pemphigoid, CREST, Celiac Disease, Cerebellar degeneration (paraneoplastic), Chronic Fatigue Syndrome, Chronic Rhinosinusitis, Chronic inflammatory demyelinating polyneuropathy, Churg Strauss Syndrome, Connective Tissue Diseases, Crohn's Disease, Cutaneous Lupus, Dermatitis Herpetiformis, Dermatomyositis, Diabetes Mellitus, Discoid Lupus Erythematosus, Drug-induced Lupus, Endocrine Orbitopathy, Glomerulonephritis, Goodpasture Syndrome, Goodpasture's Syndrome, Graves Disease, Guillian-Barre Syndrome, Miller Fisher variant of the Guillian Barre Syndrome, axonal Guillian Bane Syndrome, demyelinating Guillian Bane Syndrome, Hashimoto Thyroiditis, Herpes Gestationis, Human T-cell lymphomavirus-associated myelopathy, Huntington's Disease, IgA Nephropathy, Immune Thrombocytopenic Purpura, Inclusion body myositis, Interstitial Cystitis, Isaacs syndrome, Lambert Eaton myasthenic syndrome, Limbic encephalitis, Lower motor neuron disease, Lyme Disease, MCTD, Microscopic Polyangiitis, Miller Fisher Syndrome, Mixed Connective Tissue Disease, Mononeuritis multiplex (vasculitis), Multiple Sclerosis, Myasthenia Gravis, Myxedema, Meniere Disease, Neonatal LE, Neuropathies with dysproteinemias, Opsoclonus-myoclonus, PBC, POEMS syndrome, Paraneoplastic Autoimmune Syndromes, Pemphigus, Pemphigus Foliaceus, Pemphigus Vulgaris, Pernicious Anemia, Peyronie's Disease, Plasmacytoma/myeloma neuropathy, Poly-Dermatomyositis, Polyarteritis Nodosa, Polyendocrine Deficiency Syndrome, Polyendocrine Deficiency Syndrome Type 1, Polyendocrine Deficiency Syndrome Type 2, Polyglandular Autoimmune Syndrome Type I, Polyglandular Autoimmune Syndrome Type II, Polyglandular Autoimmune Syndrome Type III, Polymyositis, Primary Biliary Cirrhosis, Primary Glomerulonephritis, Primary Sclerosing Cholangitis, Psoriasis, Psoriatic Arthritis, Rasmussen's Encephalitis, Raynaud's Disease, Relapsing Polychondritis, Retrobulbar neuritis, Rheumatic Diseases, Rheumatoid Arthritis, Scleroderma, Sensory neuropathies (paraneoplastic), Sjogren's Syndrome, Stiff-Person Syndrome, Subacute Thyroiditis, Subacute autonomic neuropathy, Sydenham Chorea, Sympathetic Ophthalmitis, Systemic Lupus Erythematosus, Transverse myelitis, Type 1 Diabetes, Ulcerative Colitis, Vasculitis, Vitiligo, Wegener's Granulomatosis, Acrocyanosis, Anaphylactic reaction, Autoimmune inner ear disease, Bilateral sensorineural hearing loss, Cold agglutinin hemolytic anemia, Cold-induced immune hemolytic anemia, Idiopathic endolymphatic hydrops, Idiopathic progressive bilateral sensorineural hearing loss, Immune-mediated inner ear disease, and Mixed autoimmune hemolysis.

In some embodiments, a lymphocytotoxic but hematopoeitic cell-sparing high dose pulsed amount of an oxazaphosphorine drug used in the methods described herein is between 100 mg/kg and 200 mg/kg, administered daily from 1 to 7 days. In some embodiments, a lymphocytotoxic but hematopoeitic cell-sparing high-dose pulsed amount of a oxazaphosphorine drug is between 25 mg/kg and 100 mg/kg, administered daily for 4 consecutive days. In yet other embodiments, a lymphocytotoxic non-myeloablative but hematopoeitic cell-sparing high-dose pulsed amount of a oxazaphosphorine drug is 50 mg/kg administered daily for 4 consecutive days.

Exemplary oxazaphosphorine drugs include, but are not limited to, cyclophosphamide, ifosfamide, perfosfamide, trophosphamide (trofosfamide), or a pharmaceutically acceptable salt, solvate, prodrug and metabolite thereof. In some embodiments, an oxazaphosphorine drug used in the methods described herein is cyclophosphamide or a pharmaceutically acceptable salt or metabolite thereof. In some embodiments, an oxazaphosphorine drug used in the methods described herein is powdered cyclophosphamide or a pharmaceutically acceptable salt, solvate, prodrug, or metabolite thereof. In some embodiments, an oxazaphosphorine drug used in the methods described herein is lyophilized cyclophosphamide or a pharmaceutically acceptable salt, solvate, prodrug, or metabolite thereof.

Also encompassed by this disclosure is a kit for treating an autoimmune disorder including: (a) a plurality of doses of a lymphocytotoxic non-myeloablative but hematopoetic cell-sparing high-dose pulsed amount of a oxazaphosphorine drug, e.g., cyclophosphamide; (b) a plurality of doses of an effective amount of one or more monoclonal antibodies that selectively bind a B-cell specific antigen; and (b) instructions for treating the autoimmune disorder using one or more doses of the oxazaphosphorine drug and one or more doses of one or more monoclonal antibodies that selectively bind a B-cell specific antigen, where the autoimmune disorder is treated without the need for stem cell transplantation.

### DETAILED DESCRIPTION

The present invention is based, at least in part, on the discovery that administration of a lymphocytotoxic non-myeloablative amount of an oxazaphosphorine drug can be used for replacing a subject's immune cells, including autoreactive lymphocytes and immune cells associated with immune tolerance, with disease-free immune cells, without the use of stem cell transplantation. The rationale underlying this approach is the discovery that oxazaphosphorine drugs such as cyclophosphamide are lymphocytotoxic but spare hematopoietic progenitor stem cells because of high levels of aldehyde dehydrogenase, an enzyme which confers resistance to cyclophosphamide.

The present invention is also based, at least in part, on the discovery that a lymphocytotoxic but hematopoeitic cell-sparing amount of an oxazaphosphorine drug such as, for example, cyclophosphamide is effective in breaking immune tolerance which presents an obstacle in the use of various immune based therapeutics in the treatment of autoimmune disorders.

High-dose cyclophosphamide was originally used in allogeneic bone marrow transplantation because of its ability to break immune tolerance and facilitate engraftment. (See, for example, Santos et al., Transplant Proc., 4: 559-564 (1972)). However, high-dose cyclophosphamide has not been used for breaking immune tolerance associated with the use of immune therapeutics such as monoclonal antibodies that selectively bind a B-cell specific antigen, in the treatment of autoimmune disorders.

As a prodrug, cyclophosphamide is converted to 4-hydroxycyclophosphamide and its tautomer aldophosphamide in the liver. These compounds diffuse into cells and are converted into the active compound phosphoramide mustard. Alternatively, they are inactivated by the enzyme aldehyde dehydrogenase to form the inert carboxyphosphamide. Lymphoid cells, including NK cells, and B and T lymphocytes, have low levels of aldehyde dehydrogenase and are rapidly killed by high doses (i.e., lymphocytotoxic) of cyclophosphamide. In contrast, hematopoietic progenitor stem cells possess high levels of aldehyde dehydrogenase, rendering them resistant to cyclophophamide. (See, for example, Hilton, Cancer Res., 44: 5156-60 (1984); Kastan et al., Blood, 75: 1947-50 (1990); Zoumbos et al., N. Eng. J. Med., 312: 257-265 (1985); Brodsky, Sci. World J., 2: 1808-15 (2002)).

The present invention provides a method of eliminating or substantially reducing an autoimmune disorder in a subject including administering to the subject a lymphocytotoxic but hematopoeitic cell-sparing amount of an oxazaphosphorine drug, such that the subject's immune system reconstitutes without the need for stem cell transplantation and administering to the subject an effective amount of a monoclonal antibody which selectively binds a B-cell specific antigen, thereby to eliminate or substantially reduce the immune disorder.

One or more monoclonal antibodies that selectively bind a B-cell specific antigen may be administered either before the administration of a lymphocytoxic but hematopoeitic stem-cell sparing high-dose pulsed amount of an oxazaphosphorine drug such as, for example, cyclophosphamide, or one or more of antibodies that selectively bind a B-cell specific antigen may be administered after the administration of a lymphocytoxic but hematopoeitic stem-cell sparing high-dose pulsed amount of an oxazaphosphorine drug such as, for example, cyclophosphamide. In some embodiments, one or more monoclonal antibodies that selectively bind a B-cell specific antigen are administered to a subject both prior to and subsequent to the administration of a lymphocytoxic but hematopoeitic stem-cell sparing high-dose pulsed amount of an oxazaphosphorine drug such as, for example, cyclophosphamide.

In some embodiments, an effective amount of a monoclonal antibody that selectively binds a B-cell specific antigen such as, for example, rituximab, is administered to a subject having an autoimmune disorder prior to the administration of a lymphocytoxic but hematopoeitic stem-cell sparing high-dose pulsed amount of an oxazaphosphorine drug such as, for example, cyclophosphamide, thereby resulting in a synergistic effect. For example, a synergistic effect may result from the sensitization of B-cells to cytotoxic agents using a monoclonal antibody which selectively binds a B-cell specific antigen, where the B-cells are otherwise resistant to such agents, and subsequently exposing the sensitized B-cells to a cytotoxic agent, e.g., a lymphocytotoxic but hematopoeitic cell sparing high-dose pulsed amount of an oxazaphosphorine drug. Accordingly, in some embodiments, a synergistic effect can be obtained by sensitizing B-cells by using a monoclonal antibody that selectively binds a B-cell specific antigen and subsequently exposing them to a lymphocytotoxic but hematopoeitic cell sparing high-dose pulsed amount of an oxazaphosphorine drug.

In some embodiments of the present invention, additional agents and in particular agents which facilitate hematopoeitic stem cell growth such as, for example, filgrastim and pegfilgrastin, are administered to a subject following the administration of a lymphocytoxic but hematopoeitic stem-cell sparing high-dose pulsed amount of an oxazaphosphorine drug such as, for example, cyclophosphamide.

Additionally, agents such as glucocorticoids may also be administered as a part of the treatment.

### I. Definitions

In order that the present disclosure may be more readily understood, certain terms are first defined. Additional definitions are set forth throughout the detailed description.

As used herein, the phrase “high-dose pulsed amount of an oxazaphosphorine drug” refers to a non-myeloablative amount of an oxazaphosphorine drug such as, for example, cyclophosphamide, which is immunoablative, upon single or multiple dose administration to a subject (such as a human patient suffering from an autoimmune disorder), thereby resulting in a substantial reduction in or complete elimination of mature circulating lymphocytes in the subject. In some embodiments, administration of a non-myeloablative amount of cyclophosphamide results in treating, preventing, curing, delaying, reducing the severity of, ameliorating at least one symptom of an autoimmune disorder, or prolonging the survival of the subject beyond that expected in the absence of such administration. In some embodiments, “high-dose pulsed amount of an oxazaphosphorine drug” refers to a dose of cyclophosphamide administered to a subject in need thereof, which results in eliminating or substantially reducing the number of circulating lymphocytes in the subject, including those which are associated with immune tolerance, while sparing the hematopoietic progenitor stem cells. For example, in some embodiments, “high-dose pulsed amount of an oxazaphosphorine drug” is a 50 mg/kg/day dose of an oxazaphosphorine drug such as, for example, cyclophosphamide, administered to a subject in need thereof for 4 consecutive days. Cyclophosphamide is sold under common trade-names including PROCYTOX®, CYTOXAN® and NEOSAR®.

The terms “eliminating,” “substantially reducing,” “treating,” and “treatment,” as used herein, refer to therapeutic or preventative measures described herein. The methods of “eliminating or substantially reducing” employ administration to a subject having an autoimmune disorder, a lymphocytotoxic non-myeloablative amount of an oxazaphosphorine drug such as, for example, cyclophosphamide, in combination with a B-ell depleting immune therapeutic such as, for example, a monoclonal antibody that selectively binds a B-cell specific antigen.

The term “hematopoietic progenitor stem cell,” as used herein refers to any type of cell of the hematopoietic system, including, but not limited to, undifferentiated cells such as hematopoietic stem cells and progenitor cells, which are capable of reconstituting the immune system following administration of a lymphocytotoxic non-myeloablative amount of cyclophosphamide to a subject identified using the methods described herein.

The terms “B lymphocyte” and “B cell,” as used interchangeably herein, are intended to refer to any cell within the B cell lineage as early as B cell precursors, such as pre-B cells B220⁺ cells which have begun to rearrange Ig VH genes and up to mature B cells and including plasma cells. Such cells can be readily identified by one of ordinary skill in the art using standard techniques known in the art and those described herein.

The terms “immunoablation” and “immunoablative,” as used herein, refer to severe immunosuppression using a high-dose (i.e., lymphocytotoxic non-myeloablative amount) of cyclophosphamide, for example, 50 mg/kg×4 days of cyclophosphamide, which leads to substantial reduction in or elimination of the population of circulating lymphocytes, including for example, NK cells and B and T lymphocytes Immunoablation, as described herein, results in complete or substantially complete reduction in immune cells responsible for immune tolerance.

The term “lymphocytotoxic,” as used herein, refers to complete elimination of or substantial reduction in the number of circulating lymphocytes, including those associated with immune tolerance in a subject following administration of a high-dose (i.e., lymphocytotoxic non-myeloablative amount) of an oxazaphosphorine drug, such as, for example, 50 mg/kg×4 days of cyclophosphamide. The term “lymphocytotoxic,” includes killing of those immune cells by cyclophosphamide which express low levels of the enzyme aldehyde dehydrogenase.

The term “non-myeloablative,” as used herein, refers to a property of a compound such as, for example, an oxazaphosphorine drug such as cyclophosphamide, whereby the compound does not have a cytotoxic effect on myeloid cells, for example, hematopoietic progenitor stem cells. In some embodiments, a non-myeloablative agent used in the methods described herein has a cytotoxic effect on the circulating mature lymphocytes (e.g., NK cells, and T and B lymphocytes) while sparing the progenitor cells, e.g., hematopoietic progenitor stem cells that are capable of reconstituting the immune system. In some embodiments, a non-myeloablative agent used in the methods of the invention kills cells which express low levels of the enzyme aldehyde dehydrogenase (e.g., NK cells and B and T lymphocytes) while sparing cells which express high or resistant levels of the enzyme aldehyde dehydrogenase (e.g., hematopoietic progenitor stem cells).

The term “immunoglobulin” or “antibody” (used interchangeably herein) refers to a protein having a basic four-polypeptide chain structure consisting of two heavy and two light chains, said chains being stabilized, for example, by interchain disulfide bonds, which has the ability to specifically bind antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

Immunoglobulins or antibodies may be monoclonal or polyclonal and may exist in monomeric or polymeric form, for example, IgM antibodies which exist in pentameric form and/or IgA antibodies which exist in monomeric, dimeric or multimeric form. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding).

The terms “selective binding,” “specific binding,” “selectively binds,” and “specifically binds,” in the context of an antibody, mean that the antibody exhibits appreciable affinity for a particular antigen or epitope and, generally, does not exhibit significant crossreactivity. “Appreciable” or preferred binding includes binding with an affinity of at least 10⁶, 10⁷, 10⁸, 10⁹ M⁻¹, or 10¹⁰ M⁻¹. Affinities greater than 10⁷ M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant crossreactivity” is one that will not appreciably bind to an undesirable entity (e.g., an undesirable proteinaceous entity). For example, an antibody that specifically binds to CD-20 will appreciably bind CD-20 but will not significantly react with non-CD-20 proteins or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

Binding fragments are produced by recombinant DNA techniques, or by enzymatic or chemical cleavage of intact immunoglobulins. Binding fragments include Fab, Fab′, F(ab′)₂, Fabc, Fv, single chains, and single-chain antibodies. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The term “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor, L. D. et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat E. A., et al., (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or backmutation or both.

An “isolated antibody” includes an antibody that is substantially free of other antibodies having different antigenic specificities (e.g., an isolated antibody that specifically binds a B-cell specific antigen and is substantially free of antibodies or antigen-binding portions thereof that specifically bind other antigens, including other B-cell antigens). An isolated antibody that specifically binds a B-cell specific antigen may bind the same antigen and/or antigen-like molecules from other species. Moreover, an isolated antibody may be substantially free of other cellular material and/or chemicals.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The term “relapse” refers to the recurrence of an autoimmune disorder after recovery following treatment; and or recurrence of one or more symptoms associated with an autoimmune disorder after recovery following treatment. The term “remission” refers to the disappearance of autoreactive cells following treatment and/or disappearance of one or more or all symptoms associated with an adverse immune reaction, including, for example, an autoimmune disease.

### II. Exemplary Disorders

Various methods described herein can be used for treating autoimmune diseases.

Exemplary autoimmune diseases which can be treated using methods described herein include, but are not limited to, AIDS-associated myopathy, AIDS-associated neuropathy, Acute disseminated encephalomyelitis, Addison's Disease, Alopecia Areata, Anaphylaxis Reactions, Ankylosing Spondylitis, Antibody-related Neuropathies, Antiphospholipid Syndrome, Autism, Autoimmune Atherosclerosis, Autoimmune Diabetes Insipidus, Autoimmune Endometriosis, Autoimmune Eye Diseases, Autoimmune Gastritis, Autoimmune Hemolytic Anemia, Autoimmune Hemophilia, Autoimmune Hepatitis, Autoimmune Interstitial Cystitis, Autoimmune Lymphoproliferative Syndrome, Autoimmune Myelopathy, Autoimmune Myocarditis, Autoimmune Neuropathies, Autoimmune Oophoritis, Autoimmune Orchitis, Autoimmune Thrombocytopenia, Autoimmune Thyroid Diseases, Autoimmune Urticaria, Autoimmune Uveitis, Autoimmune Vasculitis, Behcet's Disease, Bell's Palsy, Bullous Pemphigoid, CREST, Celiac Disease, Cerebellar degeneration (paraneoplastic), Chronic Fatigue Syndrome, Chronic Rhinosinusitis, Chronic inflammatory demyelinating polyneuropathy, Churg Strauss Syndrome, Connective Tissue Diseases, Crohn's Disease, Cutaneous Lupus, Dermatitis Herpetiformis, Dermatomyositis, Diabetes Mellitus, Discoid Lupus Erythematosus, Drug-induced Lupus, Endocrine Orbitopathy, Glomerulonephritis, Goodpasture Syndrome, Goodpasture's Syndrome, Graves Disease, Guillain-Barre Syndrome, Guillian Bane Syndrome (Miller Fisher variant), Guillian Bane Syndrome (axonal), Guillian Bane Syndrome (demyelinating), Hashimoto's Thyroiditis, Herpes Gestationis, Human T-cell lymphomavirus-associated myelopathy, Huntington's Disease, IgA Nephropathy, Immune Thrombocytopenic Purpura, Inclusion body myositis, Interstitial Cystitis, Isaacs syndrome, Lambert Eaton myasthenic syndrome, Limbic encephalitis, Lower motor neuron disease, Lyme Disease, MCTD, Microscopic Polyangiitis, Miller Fisher Syndrome, Mixed Connective Tissue Disease, Mononeuritis multiplex (vasculitis), Multiple Sclerosis, Myasthenia Gravis, Myxedema, Meniere Disease, Neonatal LE, Neuropathies with dysproteinemias, Opsoclonus-myoclonus, PBC, POEMS syndrome, Paraneoplastic Autoimmune Syndromes, Pemphigus, Pemphigus Foliaceus, Pemphigus Vulgaris, Pernicious Anemia, Peyronie's Disease, Plasmacytoma/myeloma neuropathy, Poly-Dermatomyositis, Polyarteritis Nodosa, Polyendocrine Deficiency Syndrome, Polyendocrine Deficiency Syndrome Type 1, Polyendocrine Deficiency Syndrome Type 2, Polyglandular Autoimmune Syndrome Type I, Polyglandular Autoimmune Syndrome Type II, Polyglandular Autoimmune Syndrome Type III, Polymyositis, Primary Biliary Cirrhosis, Primary Glomerulonephritis, Primary Sclerosing Cholangitis, Psoriasis, Psoriatic Arthritis, Rasmussen's Encephalitis, Raynaud's Disease, Relapsing Polychondritis, Retrobulbar neuritis, Rheumatic Diseases, Rheumatoid Arthritis, Scleroderma, Sensory neuropathies (paraneoplastic), Sjogren's Syndrome, Stiff-Person Syndrome, Subacute Thyroiditis, Subacute autonomic neuropathy, Sydenham Chorea, Sympathetic Ophthalmitis, Systemic Lupus Erythematosus, Transverse myelitis, Type 1 Diabetes, Ulcerative Colitis, Vasculitis, Vitiligo, Wegener's Granulomatosis, Acrocyanosis, Anaphylactic reaction, Autoimmune inner ear disease, Bilateral sensorineural hearing loss, Cold agglutinin hemolytic anemia, Cold-induced immune hemolytic anemia, Idiopathic endolymphatic hydrops, Idiopathic progressive bilateral sensorineural hearing loss, Immune-mediated inner ear disease, and Mixed autoimmune hemolysis.

One of ordinary skill in the art can easily determine which diseases fall in this category, for example, by detecting auto-reactive antibodies or antibodies which react with self-antigens in a subject suffering from such a disease. Alternatively, by detecting cells in a subject which are capable of mounting an immune response against a self-antigen in the subject. Methods of diagnosing one or more autoimmune diseases encompassed by this disclosure are well-known in the art and can easily be performed by a skilled artisan.

### III. Exemplary Oxazaphosphorine Drugs

The present invention provides methods of treating autoimmune disorders using a combination of a lymphocytotoxic but hematopoeitic cell-sparing amount of an oxazaphosphorine drug and a monoclonal antibody which selectively binds to a B-cell specific antigen, without the need for stem cell transplantation.

Exemplary oxazaphosphorine drugs that may be used in the methods of the invention include, but are not limited to, for example, cyclophosphamide (CPA), ifosfamide (IFO), and trofosfamide, perfosfamide, or a pharmaceutically acceptable salt, solvate, prodrug and metabolite thereof. CPA is widely used in low to intermediate amounts as an anticancer drug, an immunosuppressant, and for the mobilization of hematopoetic progenitor cells from the bone marrow into peripheral blood prior to bone marrow transplantation for aplastic anemia, leukemia, and other malignancies. Additional oxazaphosphorine drugs that may be used in the methods of the invention include, for example, mafosfamide (NSC 345842), glufosfamide (D19575, beta-D-glucosylisophosphoramide mustard), NSC 612567 (aldophosphamide perhydrothiazine), and NSC 613060 (aldophosphamide thiazolidine).

Both CPA and IFO are prodrugs that require activation by hepatic cytochrome P450 (CYP)-catalyzed 4-hydroxylation, yielding cytotoxic nitrogen mustards capable of reacting with DNA molecules to form crosslinks and lead to cell apoptosis and/or necrosis. However, more newly synthesized oxazaphosphorine derivatives such as glufosfamide, NSC 612567 and NSC 613060, do not need hepatic activation. They are activated through other enzymatic and/or non-enzymatic pathways.

In some embodiments according to the present invention, an oxazaphosphorine drug is a lymphocytotoxic but hematopoeitic stem cell sparing high-dose pulsed amount of cyclophosphamide.

### IV. Exemplary Antibodies

In various methods of the present invention, one or more monoclonal antibodies that selectively bind a B-cell specific antigen are used in combination with a high-dose pulsed amount of an oxazaphosphorine drug for the treatment of an autoimmune disease.

B-cells are generally considered the source of all immunoglobulins and therefore, have been implicated as playing a critical role in autoimmune disorders and particularly in antibody-mediated autoimmunity such as occurring in case of rheumatoid arthritis and multiple sclerosis. The role of B-cells in autoimmune diseases was further exemplified by the generation of B-cell deficient mice. These mice were reported to be resistant to certain autoimmune diseases such as experimental autoimmune encephaliis and spontaneous insulin dependent diabetes. See, Looney, Ann. Rheum. Dis. 61:863-866 (2002).

In various embodiments of the methods of the present invention, a B-cell depleting antibody is used in combination with a lymphocytoxic but hematopoeitic cell-sparing high-dose pulsed amount of an oxazaphosphorine drug for the treatment of an autoimmune disease.

In some embodiments of the present invention, an antibody is a monoclonal antibody that specifically binds CD-20. In other embodiments, an antibody is a monoclonal antibody that specifically binds CD-22 on a B-cell. However, without wishing to be bound by theory, it is contemplated that a monoclonal antibody that selectively binds any one of B-cell specific antigens CD3d, CD5, CD6, CD9, CD19, CD20, CD21, CD22, CD23, CD24, CD27, CD28, CD37, CD38, CD40, CD45, CD46, CD48, CD53, CD69, CD70, CD72, CD73, CD79a, CD79b, CD80, CD81, CD83, CD85a, CD85d, CD85e, CD85h, CD85i, CD85j, CD85k, CD86, CD96, CD98, CD100, CD121b, CD124, CD127, CD132, CD150, CD152, CD154, CD157, CD166, CD169, CD179a, CD179b, CD180, CD185, CD196, CD197, CD205, CDw210a, CD213a1, CD257, CD267, CD268, CD269, CD274, CD275, CD276, CD278, CD279, CD300a, CD300c, CD307, CD314, CD316, CD317, CD319, CD320, CDw327, or CD331, may be used in the methods of the invention. It is also contemplated that any antibody that results in depletion or substantial reduction in the number of B-cells, or has significant activity in assays for antibody dependent cellular cytotoxicity (ADCC), such as, for example, rituximab, may be used in the methods of the invention.

Commercially available monoclonal antibodies that specifically bind B-cell specific antigens include rituximab, which binds CD-20, and epratuzumab, which binds CD-22.

Antibodies or antigen-binding portions thereof can be tested for binding to a B-cell or a B-cell specific antigen by, for example, standard assays known in the art, such as ELISA, FACS analysis and/or Biacore analysis.

Antibodies or antigen-binding portions useful in the methods of the invention may be labeled with a detectable substance using well known techniques. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹⁴C, ¹²³I, ¹²⁴I, ¹²⁵I, ¹³¹I, ^{99m}Tc, ³⁵S or ³H.

### V. Modes of Administration

The various compounds used in the methods described herein may be administered orally, parenterally (e.g., intravenously), intramuscularly, sublingually, buccally, rectally, intranasally, intrabronchially, intrapulmonarily, intraperitonealy, topically, transdermally and subcutaneously, for example. The amount of compound administered in a single dose may dependent on the subject being treated, the subject's weight, the manner of administration and the judgment of the prescribing physician. Generally, however, administration and dosage and the duration of time for which a composition is administered will approximate that which is necessary to achieve a desired result.

For example, in some embodiments, a lymphocytotoxic non-myeloablative amount of an oxazaphosphorine drug used in the methods described herein is between 100 mg/kg and 200 mg/kg, administered daily from 1 to 7 days. In other embodiments, an effective amount of a lymphocytotoxic non-myeloablative amount of an oxazaphosphorine drug is between 25 mg/kg and 100 mg/kg, administered daily for 4 consecutive days. In yet other embodiments, a lymphocytotoxic non-myeloablative amount of an oxazaphosphorine drug is 50 mg/kg administered daily for 4 consecutive days.

In general, a therapeutically effective amount of a monoclonal antibody such as, for example, an antibody that specifically binds CD-20 or CD-22, from about 0.0001 mg/Kg to 0.001 mg/Kg; 0.001 mg/kg to about 10 mg/kg body weight or from about 0.02 mg/kg to about 5 mg/kg body weight. In some embodiments, a therapeutically effective amount of a monoclonal antibody is from about 0.001 mg to about 0.01 mg, about 0.01 mg to about 100 mg, or from about 100 mg to about 1000 mg, for example.

In some embodiments, an effective amount of an antibody administered to a subject having an autoimmune disorder is between about 100 mg/m² and 200 mg/m², or between about 200 mg/m² and 300 mg/m² or between about 300 mg/m² and 400 mg/m². In a particular embodiment, an effective amount of a monoclonal antibody that selectively binds a B-cell specific antigen is about 375 mg/m².

The dose for the oxazaphosphorine drug, e.g., cyclophosphamide, for use in the methods of the present invention can be calculated according to the ideal body weight of the subject. Ideal body weight can be determined, for example, according to Metropolitan Life tables, or any other standard known in the art. If the patient's actual body weight is less than ideal, the actual weight may be used for the calculation of the oxazaphosphorine drug dose.

The optimal pharmaceutical formulations for a desired monoclonal antibody can be readily determined by one or ordinary skilled in the art depending upon the route of administration and desired dosage. (See, for example, Remington's Pharmaceutical Sciences, 18th Ed. (1990), Mack Publishing Co., Easton, Pa., the entire disclosure of which is hereby incorporated by reference).

Antibodies for use in the methods or compositions described herein can be formulated for the most effective route of administration, including for example, oral, transdermal, sublingual, buccal, parenteral, rectal, intranasal, intrabronchial or intrapulmonary administration.

In some embodiments, the present invention provides kits including one or more doses of high-dose pulsed amount of an oxazaphosphorine drug and/or one or more doses of an immune therapeutic such as, for example, a B-cell specific monoclonal antibody, packaged with instructions of use. Such instructions may pertain to use of the packaged components (i.e., one or more doses of a high-dose pulsed amount of an oxazaphosphorine drug and one or more doses of a B-cell specific monoclonal antibody) in methods of treating, preventing, ameliorating, eliminating or substantially reducing an autoimmune disorder in a patient, by administering the one or more doses of high-dose pulsed amount of an oxazaphosphorine drug and/or one or more doses of a B-cell specific monoclonal antibody.

Depending on the intended mode of administration, the compounds used in the methods described herein may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. Each dose may include an effective amount of a compound used in the methods described herein in combination with a pharmaceutically acceptable carrier and, in addition, may include other medicinal agents, pharmaceutical agents, carriers, adjuvants, diluents, etc. For example, a pharmaceutical agent may include Mesna.

Liquid pharmaceutically administrable compositions can prepared, for example, by dissolving, dispersing, etc., a compound for use in the methods described herein and optional pharmaceutical adjuvants in an excipient, such as, for example, water, saline aqueous dextrose, glycerol, ethanol, and the like, to thereby form a solution or suspension. For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; see, for example, Remington's Pharmaceutical Sciences, 18th Ed. (1990), Mack Publishing Co., Easton, Pa., the entire disclosure of which is hereby incorporated by reference).

### VI. Methods of Treatment

Methods of treatment described herein encompass methods of eliminating or substantially reducing an autoimmune disorder in a subject. All methods described herein exclude the use of stem cell transplantation.

A subject having an autoimmune disorder can be readily diagnosed based on the methods well-known in the art and those described herein, e.g., by assaying for autoreactive antibodies.

Subsequent to the diagnosis of a subject as having an autoimmune disorder, the subject can be treated using the methods described herein.

In some embodiments, a subject having an autoimmune disorder is administered a lymphocytotoxic but hematopoeitic stem cell sparing high-dose pulsed amount of an oxazaphosphorine drug, e.g., 50 mg/kg of cyclophosphamide administered each day for 4 days, and a therapeutically effective amount of a monoclonal antibody which specifically binds a B-cell specific antigen, e.g., CD-20 or CD-22.

In some embodiments, a subject having an autoimmune disorder is administered a monoclonal antibody that selectively binds a B-cell specific antigen prior to the administration of a lymphocytotoxic but hematopoeitic stem cell sparing high-dose pulsed amount of an oxazaphosphorine drug, e.g., cyclophosphamide. In other embodiments, a subject having an autoimmune disorder is administered a monoclonal antibody that selectively binds a B-cell specific antigen subsequent to the administration of a lymphocytotoxic but hematopoeitic stem cell sparing high-dose pulsed amount of an oxazaphosphorine drug, e.g., cyclophosphamide. In yet other embodiments, a subject having an autoimmune disorder is administered a monoclonal antibody that selectively binds a B-cell specific antigen both prior and subsequent to the administration of a lymphocytotoxic but hematopoeitic stem cell sparing high-dose pulsed amount of an oxazaphosphorine drug, e.g., cyclophosphamide.

In some methods of treatments, according to the invention, a method of eliminating or substantially reducing an autoimmune disorder in a subject includes (a) administering a lympocytoxic but hematopoeitic cell sparing high dose pulsed amount of an oxazaphosphorine drug, such that the subject's immune system reconstitutes without stem cell transplantation; and (b) administering a therapeutic amount of an antibody that specifically binds a B-cell specific antigen; thereby to eliminate or substantially reducing the autoimmune disorder in the subject.

The specification is most thoroughly understood in light of the teachings of the references cited within the specification which are hereby incorporated by reference. The embodiments within the specification provide an illustration of embodiments in this disclosure and should not be construed to limit its scope. The skilled artisan readily recognizes that many other embodiments are encompassed by this invention. All publications and patents cited and sequences identified by accession or database reference numbers in this disclosure are incorporated by reference in their entirety. To the extent that the material incorporated by reference contradicts or is inconsistent with the present specification, the present specification will supercede any such material. The citation of any references herein is not an admission that such references are prior art to the present disclosure.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A method for eliminating or substantially reducing an autoimmune disorder in a subject comprising administering a lymphocytotoxic but hematopoeitic stem cell sparing high-dose pulsed amount of an oxazaphosphorine drug to the subject, such that the subject's immune system reconstitutes without stem cell transplantation, and administering an effective amount of a monoclonal antibody that selectively binds a B-cell specific antigen, thereby to eliminate or substantially reduce the autoimmune disorder.

2-32. (canceled)
